(19) United States
(12) Patent Application Publication
(10) Pub. No.: US 2023/0141685 A1
(43) Pub. Date: May 11, 2023

(54) SYSTEMS AND METHODS FOR SNORING DETECTION AND PREVENTION

(71) Applicant: TA NOOMA LTD., KFAR CHABAD (IL)

(72) Inventors: BORIS MOLCHADSKY, GAN YAVNE (IL); MARK SAHAR, TEL AVIV (IL)

(21) Appl. No.: 17/920,826

(22) PCT Filed: Apr. 25, 2021
(86) PCT No.: PCT/IB2021/053398

§ 371 Date: Oct. 24, 2022

Related U.S. Application Data
(60) Provisional application No. 63/014,741, filed on Apr. 24, 2020.

(51) Int. Cl. A61B 5/00

(52) U.S. Cl. CPC A61B 5/4815; A61B 5/746; A61B 5/742; A61B 5/7455; A61B 5/7203

(57) ABSTRACT

Sleep management systems and methods detect and prevent snoring by monitoring sounds of a sleeping person. A monitoring system receives the detected snoring sound signal and generates a harmonic fingerprint of the sleeping person. A snoring score is generated and a signal is transmitted to an alert generation unit if the snoring score is above a threshold value. An alert is generated alerts to stimulate the sleeping person to transition between sleep-states enabling opening of the upper airways and reduced or stopped snoring sound.

### FIELD OF THE INVENTION

The disclosure herein relates to systems and methods for management of sleep quality. More specifically, the disclosure relates to detecting snoring sound of a person and generating alerts for its prevention.

### BACKGROUND

Sleep disorder is a medical disorder of the sleep patterns of a person or animal. Anomalies in sleep not only affect the person suffering from the sleep disturbance but also to those around them. Snoring, a sleep anomaly is the vibration of respiratory structures and the resulting sound due to obstructed air movement during breathing while sleeping. Though snoring is often considered a minor affliction, it can sometimes lead to severe health problems. Multiple studies have revealed a positive correlation between loud snoring and the risk of heart attack. Sleep apnea is also a sleep disorder where a person has pauses in breathing or periods of shallow breathing during sleep.

Currently, there is no medical treatment which can completely help with snoring or sleep apnea. Almost all treatments for snoring cater to clearing the blockage in the air passage and lessening the breathing discomfort. Medications are usually not helpful in treating snoring symptoms, though they can help control some of the underlying causes such as nasal congestion and allergic reactions.

There is a great need for a system which can detect the snoring patterns of a person and provide alerts for snoring prevention through wearable or non-wearable devices. In addition, the system should be able to differentiate between the snoring sounds and the background noise with avoidance of false positives for alert generation. The alerting system described herein comes to address this need.

### SUMMARY OF THE EMBODIMENTS

In one aspect of the invention a system is introduced for managing sleep quality of a subject. The system may comprise at least one snore detector configured and operable to detect snoring sounds and further operable to generate a snoring sound signal; and at least one monitoring system in communication with the snore detected and operable to receive the snoring sound signals from the detector and further operable to generate snoring score. Optionally, the system further comprises a display configured and operable to display the snoring score. Additionally, or alternatively, the system may include an alert generator configured to generate an alert if the snoring score is above a threshold value.

Optionally, the snore detector comprises a microphone operable to receive acoustic energy and to convert the acoustic energy into an electrical signal.

Where appropriate, the monitoring system comprises: a receiving unit configured to receive a snoring sound signal from the snoring detector; and a processor in communication with the receiving unit and operable to analyze the snoring sound signal; and a memory unit. The monitoring system may further comprise a transmission unit in communication with an alert generator and operable to send an alert signal thereto.

Variously, the alert generator, which may be configured to generate an alert if the snoring score is above a threshold value, may comprise a wearable device. The wearable device may be selected from a group consisting of wearable collars, neckbands, wristbands, armbands, wrist-watches, items of jewelry and the like as well as combinations thereof. Optionally, the alert generator may include a sleep-state intervention device positioned proximate to the subject.

The sleep-state intervention device may be operable to stimulate the subject to transition from a first sleep-state to a second sleep-state and may be selected from a group consisting of wireless speakers, vibration producing systems, buzzers, beepers, bells, bleepers, chirpers and combinations thereof.

Optionally, the system may further comprise a movement detector configured to detect movement of the subject. Variously, the movement detector is selected from a group consisting of accelerometers, gyroscopes, video cameras and the like as well as combinations thereof.

In a particular aspect of the invention, a method is taught for managing sleep quality of a subject. The method comprises providing a sleep management system; detecting a snoring sound; processing the snoring sound; and generating a snoring score for the received snoring sound. Optionally the method further includes generating an alert if the snoring score is above a threshold value. Accordingly, the method may include comparing snoring score with the threshold value and if the snoring score is above a threshold value then transmitting an alert signal to an alert generation device; and the alert generation device generating a vibration signal.

The step of generating an alert if the snoring score is above a threshold value may therefore comprise: providing a sleep-state intervention device; determining a required intervention intensity according to the snoring score; and providing intervention stimulus at the intervention intensity.

Where appropriate, the step of processing the snoring sound may comprise: generating a snoring sound signal from the snoring sound; transmitting the snoring sound signal to a monitoring system; receiving the snoring sound signal by the monitoring system; analyzing the snoring signal; generating a snoring signal fingerprint; recording the snoring signal fingerprint; and verifying the snoring sound signal.

In some examples, the method comprises displaying sleep quality indicators on a display unit. Such sleep quality indicators may be selected from a group consisting of: the snoring score, inhalation duration, exhalation duration, breath rate, amplitude of snoring sound, frequency of snoring sound, heart rate, blood pressure, body temperature, pulse rate, brain waves, ECG signal, blood oxygen measure and the like as well as combinations thereof.

Optionally, the step of detecting the snoring sound may comprise receiving acoustic energy from a direction indicative of the subject. Optionally, the step of analyzing the snoring signal comprises filtering background noise from the snoring sound.

Additionally or alternatively, the step of generating a snoring signal fingerprint comprises identifying sleep quality indicators selected from a group consisting of: the snoring score, inhalation duration, exhalation duration, breath rate, amplitude of snoring sound, frequency of snoring sound, heart rate, blood pressure, body temperature, pulse rate, brain waves, ECG signal, blood oxygen measure and combinations thereof.

In one aspect of the invention, a system for detecting and preventing snoring sounds is disclosed. The system includes a microphone or detector and sleep sensors for detecting snoring sounds and other physiological parameters of a sleeping person. A monitoring system receives the detected snoring sound signal and physiological parameters from the microphone and sleep sensors and processes the received signals and generate harmonic fingerprinting of sound of the sleeping person. The monitoring system also generates a snoring score from the received signals and compares it with a threshold value. A signal is transmitted to an alert generation unit in case the snoring score is above the threshold value. The alert generation unit generate alerts in form of vibration or sound signal and causes the sleeping person to shift to a lighter sleep stage, change sleeping posture/position and enable opening of the upper airways. The snoring score and the measured parameters are displayed on a display unit of the monitoring system.

In another aspect of the invention, a method for detecting and preventing snoring sounds is disclosed. The method includes detecting snoring sounds and other physiological parameters of a sleeping person and transmitting the detected signals to a monitoring system. The method further includes processing the received signals and generating harmonic fingerprinting of sound of the sleeping person. A snoring score is also generated from the received signals and compared with a threshold value. The method also includes transmitting a signal to an alert generation unit in case the snoring score is above the threshold value. The alerts are generated in form of vibration or sound signal which causes the sleeping person to shift to a lighter sleep stage, change sleeping posture/position and enable opening of the upper airways. The method further includes displaying the snoring score and the measured parameters on a display unit of the monitoring system.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice.

As used in this specification, the singular indefinite articles “a”, “an”, and the definite article “the” should be considered to include or otherwise cover both single and plural referents unless the content clearly dictates otherwise. In other words, these articles are applicable to one or more referents. As used in this specification, the term “or” is generally employed to include or otherwise cover “and/or” unless the content clearly dictates otherwise.

In the accompanying drawings:

FIG. 1 illustrates a schematic view of a sleep quality management system 100 for detecting and preventing sleep disorders;

FIG. 2 illustrates a block diagram of system components of a monitoring system 106; and

FIGS. 3A and 3B (collectively FIG. 3) illustrate a flowchart 300 representing method steps for detecting snoring sounds and generating alerts.

### DESCRIPTION OF THE SELECTED EMBODIMENTS

Aspects of the present disclosure relate to system and methods for a sleep quality management system for detecting sleeping disorders and generating alerts for its prevention.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally, or alternatively, the data processor may access a non-volatile storage, for example, a magnetic hard-disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

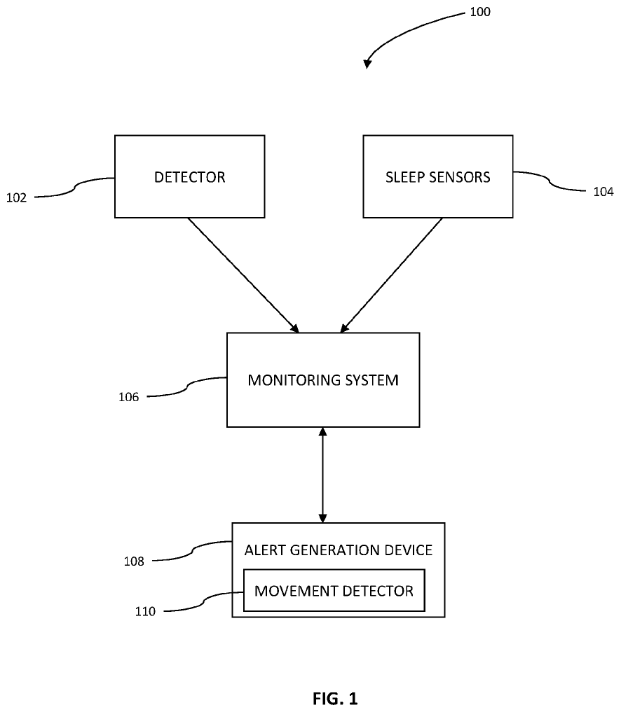

FIG. 1 illustrates a schematic of a sleep quality management system 100 for detecting sleeping disorders and generating alerts for its prevention. The sleeping disorders include snoring, obstructive sleep apnea (OSA), bruxism (teeth grinding), etc. In a particular embodiment, the system 100 includes a microphone or detector 102 for detecting the snoring sound of a person (not shown in FIG. 1). The microphone or detector 102 is configured to receive acoustic energy or acoustic signals indicative of snoring sounds from a sleeping person. The microphone or detector 102 is preferably placed near the face of the sleeping person for detecting the snoring sound. The microphone or detector 102 can be placed on the bed near the pillow of the sleeping person. Alternatively, the microphone or detector 102 can be placed on any other object, like a table within a prescribed range from the face of the person. In a preferred embodiment, the microphone or detector 102 is placed within a distance of 1 meter from the face of the person. Any other suitable placement of microphone or detector 102 can be chosen without limiting the scope of the invention.

The sleep quality management system 100 is shown to include one microphone or detector 102. In an alternative embodiment of the invention, the sleep quality management system 100 can include two or more sound detectors for detecting the snoring sound of a person. A number of other sleep sensors 104 can also be placed near the sleeping person to detect his/her health parameters. The sleep sensors 104 include, but are not limited to, a heart rate sensor, a blood pressure sensor, a temperature sensor, a brain activity sensor, an ECG sensor, a blood oxygen sensor, electrodermal activity and so on. Where appropriate, sleep sensors may be provided in a wristband, or other wearable item and may include optical sensors for measuring blood pressure or for checking blood oxygen concentration. These sleep sensors 104 may continuously measure the various health parameters of the sleeping person and generate appropriate alerts in case major abnormalities are detected.

The snoring sounds detected by the microphone or detector 102 and other health parameters detected by sleep sensors 104 are transmitted to a monitoring system 106 of the sleep quality management system 100. In a preferred embodiment of the sleep quality management system 100, the microphone or detector 102 and the sleep sensors 104 are connected to the monitoring system 106 through a Bluetooth network. However, any other communication network can be used for the purpose, including a Wired LAN, a Wireless LAN, a WiFi Network, a Zigbee Network, a Z-Wave Network or an Ethernet Network. The communication networks disclosed above are exemplary in nature and should not limit the scope of the invention. The monitoring system 106 is responsible for analyzing the received snoring sounds and generating appropriate alerts through a wearable or non-wearable alert generation device 108 for its prevention as will be explained below.

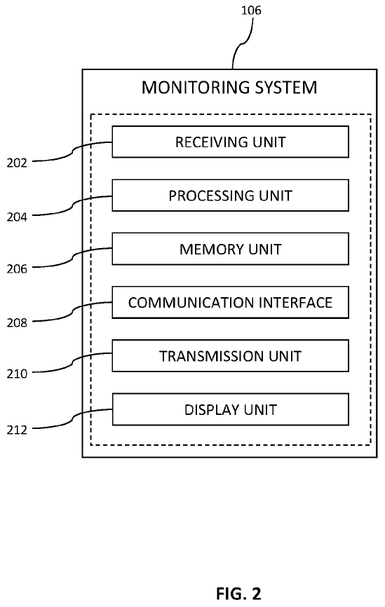

FIG. 2 illustrates a block diagram of selected system components of the monitoring system 106. A receiving unit 202 receives the snoring sound signal from the microphone or detector 102. The snoring sound signal is received in form of acoustic energy or acoustic signals indicative of snoring sounds from the sleeping person. The receiving unit 202 also receives the heath parameters detected by various sleep sensors 104. The various health parameters include, but are not limited to, a measure of heart rate, blood pressure, body temperature, pulse rate, brain waves, ECG signal, blood oxygen measure, etc. of the sleeping person.

The system 106 includes a processing unit 204 which analyzes and processes the signals received by the receiving unit 202. The processing unit 204 firstly, filters out the background noise from snoring sound signal for avoidance of false positive. The background noise may include the sound of an object like a wall clock, electrical equipment like air conditioner, or any other stationary, moving or vibrating object present within the room or outside the room. The processing unit 204, then measures the frequency and amplitude (intensity) of the snoring sound signal. The processing unit 204 may also measure the following parameters, amongst others:
i. Inhalation and Exhalation duration
ii. Breath rate
iii. Amplitude and frequency of snoring sound
iv. Other physiological parameters such as the heart rate, blood pressure, body temperature, pulse rate, brain waves, ECG signal, blood oxygen measure, etc.

A harmonic fingerprinting of the snoring sound of the person is developed using the measured parameters of the frequency and amplitude (intensity) of the snoring sound signal, inhalation/exhalation ratio and the breath rate along with their occurrence timings during the sleep. The harmonic fingerprinting represents a snoring pattern of the person during various times of the sleep. In a preferred embodiment, the harmonic fingerprinting of the snoring sound is represented in form of Amplitude/Frequency profile. Alternatively, the harmonic fingerprinting can be represented in form of ratio of various parameters discussed above without limiting the scope of the invention. The harmonic fingerprinting of the person is stored in a memory unit 206 of the system 106. The harmonic fingerprinting can be used by medical doctors for advising curing and precautionary measures for reducing snoring.

The memory unit 206 may be defined as a repository of all the information received from the processing unit 204 and also received by the receiving unit 202. The memory unit 206 may include a main memory, such as a Random Access Memory (RAM) or other dynamic storage device. The main memory may be used for storing temporary variables or other intermediate information during storing of the events information. The memory unit 206 may further include a Read Only Memory (ROM) (or other non-volatile memory) or other static storage device for storing static information and instructions of the events. The memory unit 206 can further be a storage device such as a magnetic disk or optical disk, a hard disk drive (HDD) for reading from and writing to a hard disk, a magnetic disk drive for reading from and writing to a magnetic disk, and/or an optical disk drive (such as DVD) for reading from and writing to a removable optical disk.

The processing unit 204 also processes the snoring sound signal and actuates the alert generation device 108 for generating various alerts for snoring prevention. In an exemplary embodiment, one or more of the following parameters are considered for alert generation:
i. Inhalation and Exhalation duration
ii. Breath rate
iii. Amplitude and frequency of snoring sound
iv. Other physiological parameters such as the heart rate, blood pressure, body temperature, pulse rate, brain waves, ECG signal, blood oxygen measure, etc.

A score is given to each of the parameters listed above and a snoring score is calculated using a combination of one or more of the parameters. The calculated snoring score is compared with a predetermined threshold value. The sound signal is considered as acceptable snoring sound if the snoring score is less than the threshold value and no alert generation signal is sent to the alert generation device 108. In case the snoring score is equal to or above the threshold value, an alert generation signal is transmitted to the alert generation device 108 through a communication interface 208 or a transmission unit 210 of the monitoring system 106. In a preferred embodiment of the present invention, the transmission unit 210 transmits the signal to the alert generation device 108 through a Bluetooth network. However, any other communication network can be used for the purpose, including a Wired LAN, a Wireless LAN, a WiFi Network, a Zigbee Network, a Z-Wave Network or an Ethernet Network.

It is further noted that in various embodiments the transmission unit 210 and communication interface 208 are configured to communicate data gathered by the monitoring system to a computer network such as the internet wherein the data may recorded in a database preferably protected by security systems such as a blockchain technology or the like such that the privacy of the subject is maintained.

As the data is collected, it will be appreciated that machine learning systems may be adapted to improve with use according to the accumulated data.

As an exemplary embodiment of the present invention, the following conditions can be verified for alert generation:
i. Inhalation/Exhalation ratio—Typically between 1:1 and 1:6. Breath rate maybe identified by a shorter inhalation phase˜1 s followed by a longer exhalation phase˜6-10 s
ii. Frequency of sound between 5-10 Hz

It should be clearly understood to a person skilled in the art that any other parameter or values can be considered for alert generation.

The various parameters measured for calculating the snoring score and the calculated snoring score are stored in the memory unit 206 and associated with the harmonic fingerprinting of the person. The measured parameters and the snoring score can also be displayed on a display unit 212 of the monitoring system 106.

The alert generation device 108 can be a wearable or a non-wearable device. The wearable device 108 can be worn or attached to the body parts of the sleeping person, for example, on the wrist, around the neck, around stomach belly, on the ankle, etc. The wearable device 108 is not so limited and can be worn, attached, or otherwise disposed adjacent to any limb or portion of the sleeping person. Exemplary wearable device 108 can be in form of a wearable collar, a neckband, a wristband, an armband, etc. The wearable device 108 can also be integrated with a wrist-watch or a jewelry worn by the sleeping person. The non-wearable device 108 can include wireless speakers, vibration producing systems, etc. placed near the sleeping person.

The alert generation device 108 produces alerts in form of vibration signals. Alternatively, alerts can be in form of sound signals consisting of a buzzer, a beeper, a bell, a bleeper, a chirper and combinations thereof. The audio/vibration means provided above for generating alerts are exemplary in nature and should not limit the scope of the invention. The intensity and frequency of the vibration or sound signal is dependent upon the snoring score calculated by the processing unit 204. In a preferred embodiment, the intensity and frequency of the vibration or sound signal is directly proportional to the snoring score. However, the intensity and frequency of the vibration or sound signal should be controlled to avoid awakening the sleeping person, unless an emergency is detected. Thus the intesity of the alert signal may be selected or calculated in real time according to monitored parameters.

Since the objective of the invention is prevention of the snoring disorder, the generated alerts should help the sleeping person to reduce or stop snoring. Accordingly, a sleep-state intervention device may be provided to produce an intervention stimulus to stimulate the subject to transition from a first sleep-state to a second sleep-state. For example, an intervention stimulus such as a vibration or sound signal may cause the sleeping person to shift to a lighter sleep stage, change sleeping posture/position and enable opening of the upper airways. This can result in reduced or stopped snoring. The alert generation unit 108 includes a movement detector 110 which is configured to detect a movement or motion of the sleeping person. The movement detector 110 may comprise one or more sensors (e.g., accelerometers, gyroscopes, video cameras etc.) to detect movement signals caused by movement of the sleeping person. The determination of movement of the sleeping person is used to automatically increase or decrease the intensity and frequency of the vibration or sound signal.

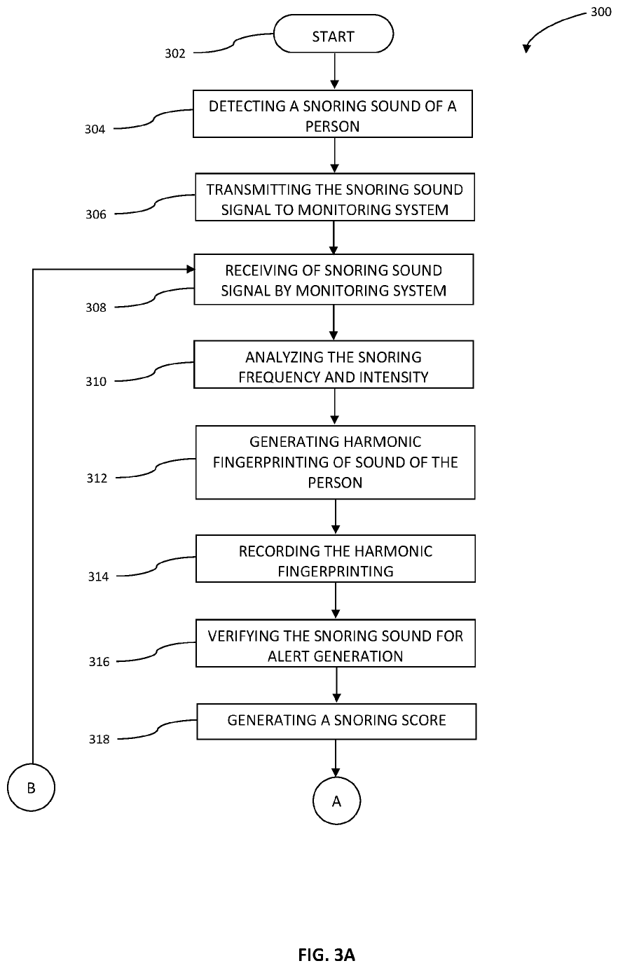

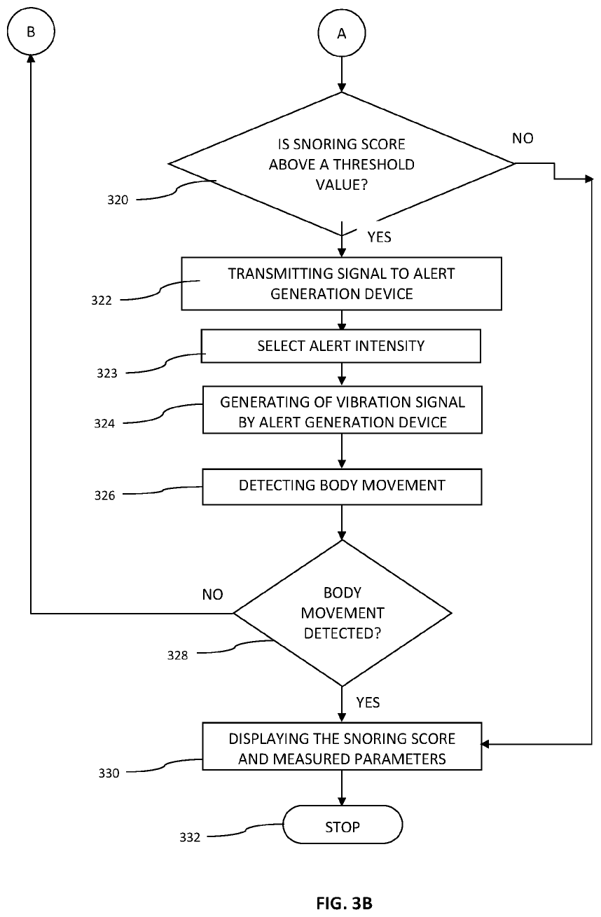

FIG. 3 illustrates a flowchart 300 representing method steps for detecting snoring sounds and generating alerts. The process starts at step 302 and snoring sound of a person is detected by the microphone or detector 102 at step 304. The snoring sound signal is transmitted to the receiving unit 202 of the monitoring system 106 at step 306. The signal received at step 308 is analyzed by the processing unit 204 at step 310 for various parameters including the frequency and intensity of the signal. At step 312, the harmonic fingerprinting of the sound of the sleeping person is generated using parameters like inhalation and exhalation duration, breath rate, amplitude and frequency of snoring sound, other physiological parameters such as the heart rate, blood pressure, body temperature, pulse rate, brain waves, ECG signal, blood oxygen measure, electrodermal activity etc. Accordingly, sleep sensors may be provided such as optical sensors for measuring blood pressure or blood oxygen concentration.

The harmonic fingerprinting of the person is recorded at step 314 in the memory unit 206. At step 316, the snoring sound is verified for alert generation by generating a snoring score at step 318 using the above mentioned parameters. At step 320 the snoring score is compared with a threshold value. In case, it is determined that the snoring score is equal to or above the threshold value, a signal is transmitted at step 322 to the alert generation device 108.

The alert generation device may be configured and operable to calculate or otherwise select a required alert intensity according to monitored parameters 323. For example the system may communicate with a computing device such as a smart phone, tablet or the like which may perform the required calculations as required.

At step 324, the alert generation device 108 generates alerts in form of a vibration or sound signal or a combination thereof. In case, it is determined at step 320 that the snoring score is below the threshold value, no signal is transmitted to the alert generation device 108. The process then jumps to step 330 and the measured parameters and the snoring score are displayed on the display unit 212.

After the generation of vibration signal by the alert generation device 108 at step 324, a body movement of the sleeping person is detected at step 326 by the movement detector 110 of the alert generation device 108. In case the body movement is detected at step 328, the process goes to step 330 to display the measured parameters and the snoring score are displayed on the display unit 212. In case the body movement is not detected at step 328, the process goes to step 308 and subsequent steps are repeated as described above. The process stops at step 332.

The system 100 is advantageous over the existing technologies as it detects the snoring sounds of a sleeping person with minimal or zeroed false positives.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1. A method for managing sleep quality of a subject, the method comprising:
providing a sleep management system;
detecting a snoring sound;
processing the snoring sound; and
generating a snoring score for the received snoring sound.

2. The method of claim 1 further comprising:
generating an alert if the snoring score is above a threshold value.

3. The method of claim 1 wherein the step of processing the snoring sound comprises:
generating a snoring sound signal from the snoring sound;
transmitting the snoring sound signal to a monitoring system;
receiving the snoring sound signal by the monitoring system;
analyzing the snoring signal;
generating a snoring signal fingerprint;
recording the snoring signal fingerprint; and
verifying the snoring sound signal.

4. The method of claim 2 wherein the step of generating an alert if the snoring score is above a threshold value comprises:
comparing snoring score with the threshold value;
if the snoring score is above a threshold value then:
transmitting an alert signal to an alert generation device; and
the alert generation device generating a vibration signal.

5. The method of claim 3 further comprising displaying sleep quality indicators on a display unit.

6. The method of claim 5 wherein the sleep quality indicators are selected from a group consisting of: the snoring score, inhalation duration, exhalation duration, breath rate, amplitude of snoring sound, frequency of snoring sound, heart rate, blood pressure, body temperature, pulse rate, brain waves, ECG signal, blood oxygen measure and combinations thereof.

7. The method of claim 3 wherein the step of detecting the snoring sound comprises receiving acoustic energy from a direction indicative of the subject.

8. The method of claim 3 wherein the step of analyzing the snoring signal comprises filtering background noise from the snoring sound.

9. The method of claim 3 wherein the step of generating a snoring signal fingerprint comprises identifying sleep quality indicators selected from a group consisting of: the snoring score, inhalation duration, exhalation duration, breath rate, amplitude of snoring sound, frequency of snoring sound, heart rate, blood pressure, body temperature, pulse rate, brain waves, ECG signal, blood oxygen measure and combinations thereof.

10. The method of claim 2 wherein the step of generating an alert if the snoring score is above a threshold value comprises:
providing a sleep-state intervention device;
determining a required intervention intensity according to the snoring score; and
providing intervention stimulus at the intervention intensity.

11. A system for managing sleep quality of a subject, the system comprising:
at least one snore detector configured and operable to detect snoring sounds and further operable to generate a snoring sound signal; and
at least one monitoring system in communication with the snore detected and operable to receive the snoring sound signals from the detector and further operable to generate snoring score.

12. The system of claim 11, further comprising a display configured and operable to display the snoring score.

13. The system of claim 11, further comprising an alert generator configured to generate an alert if the snoring score is above a threshold value.

14. The system of claim 11, wherein the snore detector comprises a microphone operable to receive acoustic energy and to convert the acoustic energy into an electrical signal.

15. The system of claim 11 wherein the monitoring system comprises:
a receiving unit configured to receive a snoring sound signal from the snoring detector;
and
a processor in communication with the receiving unit and operable to analyze the snoring sound signal; and
a memory unit.

16. The system of claim 15 wherein the monitoring system further comprises a transmission unit in communication with an alert generator and operable to send an alert signal thereto.

17. The system of claim 11, further comprising an alert generator configured to generate an alert if the snoring score is above a threshold value wherein said alert generator comprises a wearable device.

18. The system of claim 17 wherein the wearable device is selected from a group consisting of wearable collars, neckbands, wristbands, armbands, wrist-watches, items of jewelry and combinations thereof.

19. The system of claim 11, further comprising an alert generator configured to generate an alert if the snoring score is above a threshold value wherein said alert generator comprises a sleep-state intervention device positioned proximate to the subject said sleep-state intervention device operable to stimulate the subject to transition from a first sleep-state to a second sleep-state.

20. The system of claim 19 wherein the sleep-state intervention device is selected from a group consisting of wireless speakers, vibration producing systems, buzzers, beepers, bells, bleepers, chirpers and combinations thereof.

21. The system of claim 11 further comprising a movement detector configured to detect movement of the subject.

22. The system of claim 21 wherein the movement detector is selected from a group consisting of accelerometers, gyroscopes, video cameras and combinations thereof.
